(19) United States
(12) Patent Application Publication
(10) Pub. No.: US 2019/0038191 A1
(43) Pub. Date: Feb. 7, 2019

(54) GLUCOSE SENSORS AND METHODS FOR DETECTING GLUCOSE IN BODILY FLUIDS

(71) Applicant: ZANSORS LLC, Arlington, VA (US)

(72) Inventors: Gene P. Parunak, Saline, MI (US); Aaron M. Kehrer, Ypsilanti, MI (US)

(73) Assignee: ZANSORS LLC, Arlington, VA (US)

(21) Appl. No.: 16/056,028

(22) Filed: Aug. 6, 2018

Related U.S. Application Data
(60) Provisional application No. 62/541,999, filed on Aug. 7, 2017.

(51) Int. Cl. A61B 5/145; A61B 5/1464; A61B 5/1486

(52) U.S. Cl. CPC A61B 5/14532; A61B 5/157; A61B 5/1486; A61B 5/1464

(57) ABSTRACT

A glucose sensor may include a substrate, a processor mounted to the substrate, and a plurality of electrodes mounted to the substrate and operatively connected to the processor. The plurality of electrodes may be adapted to detect a condition on or below a surface.

### CROSS REFERENCE TO RELATED APPLICATION

This application claims priority from U.S. Provisional Application No. 62/541,999 filed Aug. 7, 2017, the entirety of which is incorporated herein by reference.

### BACKGROUND OF THE INVENTION

### Technical Field

The present invention provides enhancements in medical devices, in particular, improvements in devices and methods for detecting and/or monitoring glucose levels in bodily fluids, such as in blood.

### Description of Related Art

The detection or monitoring of the components of bodily fluids, such as blood, can be critical to establishing or maintaining a patient's health or condition. For instance, the presence or relative concentration of glucose in the blood stream can be critical to monitoring the health of diabetics, pre-diabetics, and infants, especially, neonates, whose blood glucose homeostasis is typically monitored to detect any evidence of hypoglycemia or hyperglycemia.

It is conventional practice in neo-natal intensive care units (NICUs) to monitor a neonate's blood glucose levels. Neonates in NICUs typically require glucose testing every three hours or 9 times per day. Conventional glucose testing in neonates typically is practiced with a “needle prick” of the “warmed” heel of a neonate, and with a bedside reagent test-strip of the blood sample taken. The blood-laden test strip is then used with a glucose analyzer (for example, a glucose analyzer employing reflectance, colorimeter, or electrode methods) to detect the glucose content of the neonate's blood.

Each heel stick (which extracts approximately 0.2 cubic centimeters [cc] or 200 microliters [μL] of blood) understandably can cause considerable pain and stress to the neonate, who often has underdeveloped skin and tissue. In addition, the neonate in the NICU typically may have limited fluids, including limited blood supply, which can be utilized for glucose screening.

The lack of real-time glucose monitors in neonates, and others, can be a major unmet need. Specifically, there is a need in monitoring glucose in neonates for reducing sample volume collection and reducing the number of needle pricks, among other things.

Aspects of the present invention, specifically, the sensors, devices, and methods, provide means for addressing this unmet need in the NICU, and in other glucose sampling environments.

### SUMMARY OF THE INVENTION

Aspects of the present invention may provide sensors, devices, and methods for detecting and/or monitoring conditions and substances on or below the skin of a subject, for example, a neonate. In some aspects, the substance detected may be glucose. However, as disclosed herein, many different types of substances and conditions may be monitored and/or detected by one or more embodiments of the invention, in their various aspects.

Some embodiments of the invention may include a glucose sensor, for example, a substantially continuously monitoring glucose (CMG) sensor, comprising or consisting of a substrate; a processor mounted to the substrate; a plurality of electrodes mounted to the substrate and operatively connected to the processor, the plurality of electrodes adapted to detect a condition on or below a surface. In some aspects, each of the plurality of electrodes may comprise at least one projection, for example, at least one micro-needle; however, a plurality of micro-needles may be provided.

In some aspects, each of the plurality of electrodes may include a cylindrical body, for example, an electrically conductive cylindrical body, and the cylindrical body may include at least one projection, for example, at least one micro-needle. The cylindrical body may be a circular cylindrical body, an elliptical cylindrical body, or a polygonal cylindrical body, among other shapes. In other aspects, the body may not be cylindrical.

In some aspects, each of the plurality of electrodes may comprise an elastomeric collar, insert, or annulus about or retaining the cylindrical body. The elastomeric collar may be made from thermoplastic elastomer, a thermoplastic urethane, or a liquid silicone rubber.

In some aspects, the sensor may include a potentiostat mounted to the substrate, and the potentiostat may be electrically coupled to the plurality of electrodes.

In some aspects, the plurality of electrodes may be distally mounted to the substrate. For example, the plurality of electrodes may be distally mounted to the substrate by a flexible projection, for example a flexible, electrically conductive projection.

In some aspects, the glucose sensor may be a neonatal glucose sensor; however, aspects of the invention may be used for any human or animal subject or patient.

Some embodiments of the invention may include a method for detecting glucose (or other substances and/or conditions) in a subject. The method may comprise contacting the skin of the subject with at least two electrically conductive projections or micro-needles; sensing an electrical signal between the at least two electrically conductive projections; relating the sensed electrical signal to a glucose concentration; and transmitting the glucose concentration to a receiver. In some aspects, the method may include allowing at least one of the at least two electrically conductive projections to deflect when contacting the skin of the subject. For example, in some aspects, mounting the electrically conductive projections in an elastomeric collar may allow the electrically conductive projections to deflect. In some aspects, allowing the electrically conductive projections to deflect may be practiced by mounting the electrically conductive projections on a flexible projection, for example, on a flexible “finger.”

In some aspects, the method may include collecting a fluid sample from the subject. For example, in some aspects, the electrically conductive projections, or micro-needles, may be hollow projections, and the fluid sample may be collected by collecting fluid through the hollow projections.

These and other aspects, features, and advantages of the invention will become apparent from the following detailed description of the various aspects of the invention taken in conjunction with the accompanying drawings.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention will be readily understood from the following detailed description of aspects of the invention taken in conjunction with the accompanying drawings in which:

FIG. 1 is a perspective view of a glucose sensor according to some aspects of the invention.

FIG. 2 is a top plan view of the glucose sensor shown in FIG. 1.

FIG. 3 is a side elevation view of the glucose sensor shown in FIG. 1.

FIG. 4 is an exploded perspective view of the glucose sensor shown in FIG. 1.

FIG. 5 is a bottom view of the substrate shown in FIG. 4 according to some aspects of the invention.

FIG. 6 is a detailed view of the substrate shown in FIG. 5 as identified by Detail 6 in FIG. 5.

FIG. 7 is a detailed cross section of the electrode assembly shown in FIGS. 5 and 6 as viewed along section lines 7-7 in FIG. 6, according to some aspects of the invention.

FIG. 7A is a top plan view of the electrode assembly shown in FIG. 7 according to some aspects of the invention.

FIGS. 8 through 17 are perspective views of cylindrical bodies that may be used for electrodes according some aspects of the invention.

FIGS. 18 and 19 are top plan views of the electrode cylindrical bodies engaged with a substrate according to some aspects of the invention.

FIG. 20 is a detailed cross section of, similar to FIG. 7, of another electrode assembly according to some aspects of the invention.

FIG. 21 is a side elevation view, similar to FIG. 7, of another electrode assembly according to some aspects of the invention.

FIG. 22 is a side elevation view of another glucose sensor according to some aspects of the invention.

FIG. 23 is a bottom perspective view of the glucose sensor shown in FIG. 22 with the cover removed.

FIG. 24 is a detailed view of the electrode assembly shown in FIG. 23 as identified by Detail 24 in FIG. 23.

FIG. 25 is a detailed cross section view of the electrode assembly shown in FIGS. 23 and 24 as viewed along section lines 25-25 shown in FIG. 24, according to some aspects of the invention.

### DETAILED DESCRIPTION OF THE INVENTION

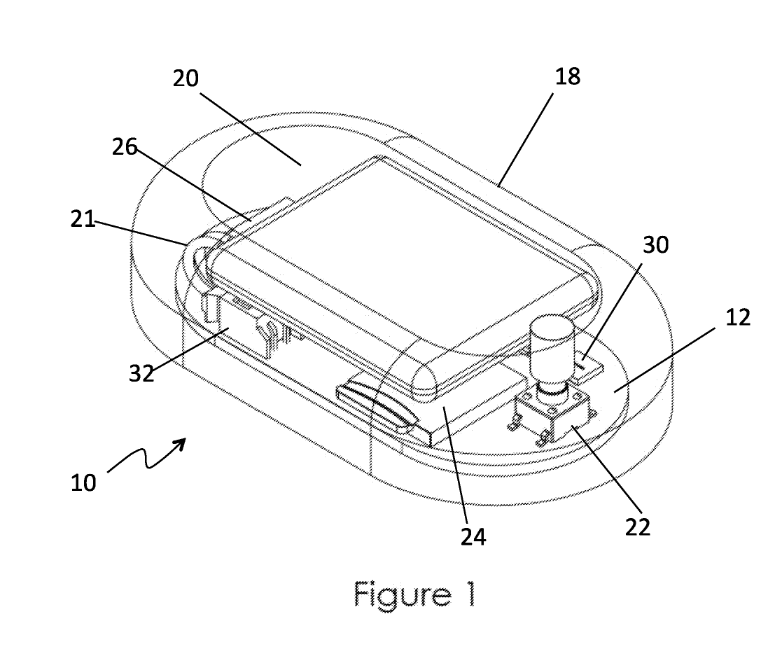

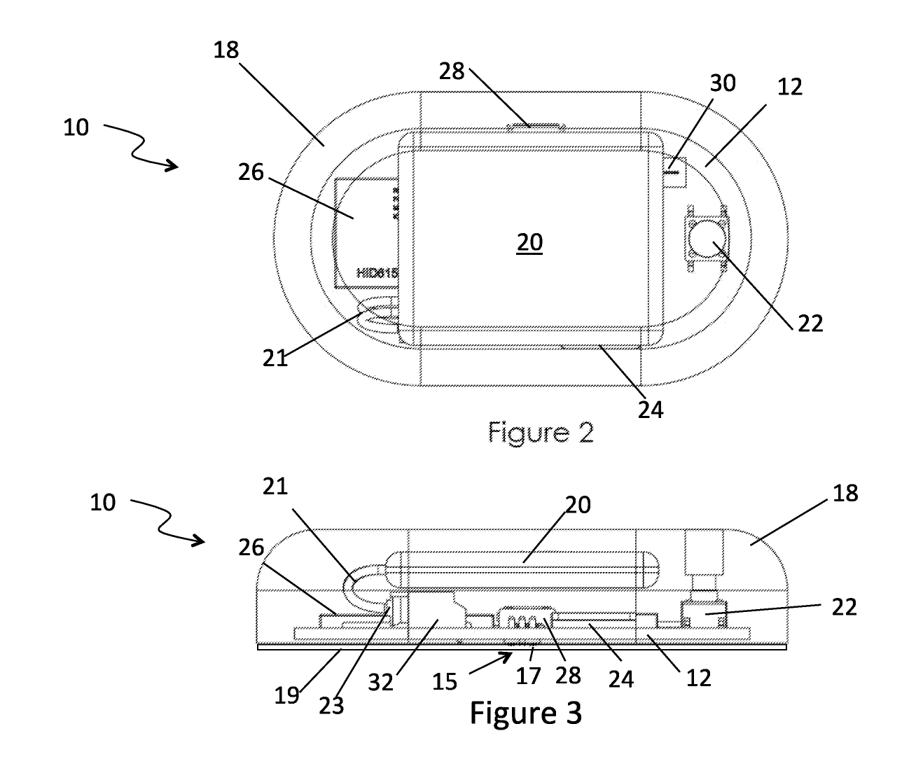

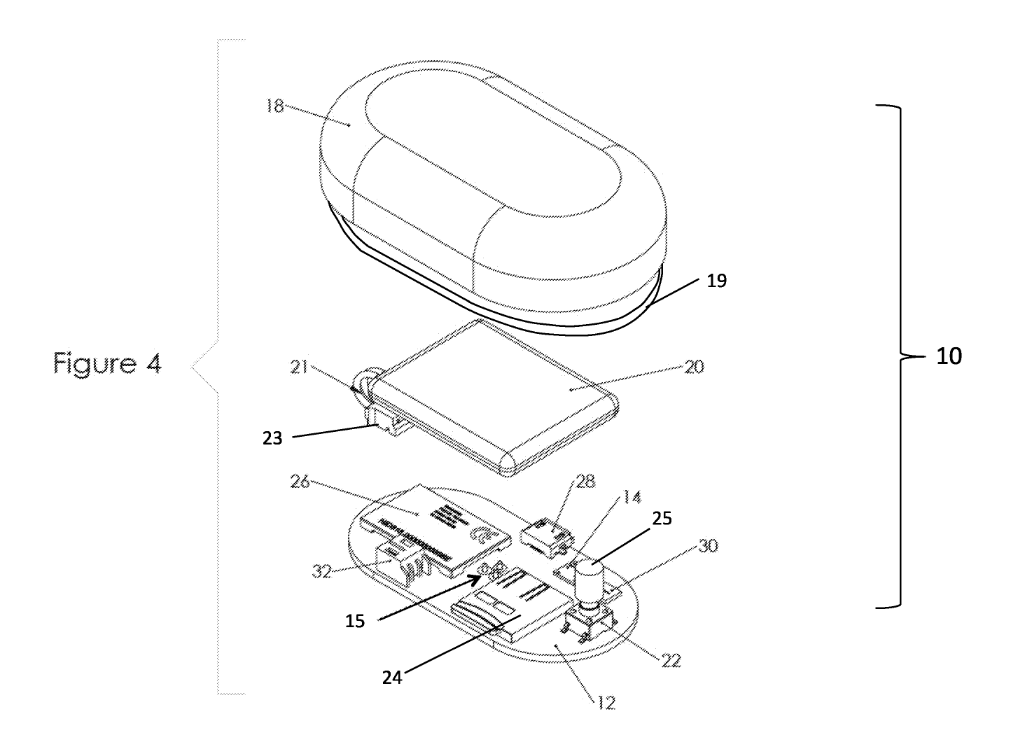

FIG. 1 is a perspective view of a glucose sensor 10 according to some aspects of the invention. FIG. 2 is a top plan view of glucose sensor 10 and FIG. 3 is a side elevation view of glucose sensor 10 shown in FIG. 1. FIG. 4 is an exploded perspective view of glucose sensor 10 shown in FIG. 1.

As shown in FIGS. 1 through 4, according to some aspects, glucose sensor 10 may include a substrate 12, one or more processors 14 mounted to substrate 12, and an electrode assembly 15 having a plurality of electrodes 16, for example, having projections or “micro-needles” 17, where electrode assembly 15 is mounted to the substrate 12 and operatively connected to the one or more processors 14. In some aspects, substrate 12 may be any substrate adapted to receive one or more processors 14 and electrode assembly 15. For example, in some aspects, substrate 12 may comprise a printed circuited board (PCB) assembly, for example, a PCB assembly provided by a conventional PCB supplier. In some aspects, substrate 12 may comprise any metallic or non-metallic material, for example, a plastic or a cellulose-based substrate (such as a paper substrate), among other non-metallic materials. As discussed further below, in some aspects, substrate 12 may comprise at least some elastomeric material, for example, a portion of substrate 12 about electrodes 16 may comprise an elastomeric material. According to some aspects of the invention, sensor 10 (and any sensor disclosed herein) may be adapted to continuously detect or sense glucose (or any substantive disclosed herein) or be adapted to intermittently detect or sense glucose (or any substance disclosed herein).

The one or more processors 14 may comprise any processor adapted to execute software and adapted to receive and transmit electrical signals, for example, generated by electrode assembly 15, or from any other components mounted on substrate 12. For example, processor 14 may be a microprocessor provided by STMicroelectronics, for example, an STM32L0 microprocessor, or its equivalent.

The electrode assembly 15 of glucose sensor 10 may include one or more electrodes 16, where each of the electrodes 16 may include projections or micro-needles 17 adapted to perform the functions disclosed herein. For example, in some aspects, the one or more micro-needles 17 in electrode assembly 15 may by adapted to contact and/or at least partially penetrate a surface, for example, the skin of a subject, such as a human or animal subject, and detect the presence of a target substance and/or a target condition present on and/or below the surface. In some aspects, the surface contacted or penetrated by projections or micro-needles 17 may be a “target surface.” For example, in some aspects, electrode assembly 15 may be adapted to detect glucose as the target substance, among other substances, and/or to detect a voltage differential or electric current as a target condition, among other conditions, on, in, or beneath a target surface. In some aspects, the voltage differential or electric current detected may be associated with a glucose content of the fluid, for example, the concentration of glucose in the fluid.

In some aspects, the one or more projections or micro-needles 17 in electrodes 16 may be adapted to detect a target substance and/or a target condition in a fluid, for example a gas or a liquid, present on the surface or beneath the surface, for example in the blood or in the “interstitial fluid” present beneath the surface of the skin of a subject.

In some aspects, various physiological measurements may be sensed or detected in addition to or in place of, for example, glucose content, including electrocardiography (EKG), electromyography (EMG), bio-impendence, and the like. For example, in some aspects, projections or micro-needles 17 may be used to detect glucose concentration, for example in blood, and cardiovascular events (for example, myocardial infarction (MI) or angina). The detection of glucose may alternate with the detection or monitoring of cardiovascular function, or the detection of glucose may be practiced effectively simultaneously with the detection or monitoring of cardiovascular function.

According to some aspects of the invention, projections or micro-needles 17 may be adapted to extract at least some fluid, for example, blood or interstitial fluid, from below the surface of the skin and transmit the fluid. In some aspects, the fluid extracted may be forwarded to a detection and/or analysis device, for example, for colorimetric, electrochemical, ultrasound, dielectric spectroscopy, or near infrared analysis, to detect the presence of glucose and/or other substances, as disclosed herein. In some aspects, the presence or content of glucose may be detected by an electrochemical analysis of the fluid, for example, by potentiometric, amperometric, or conductometric analysis. In one aspect, the presence or content of glucose may be detected based upon interaction of one or more components of the fluid with an enzyme, for example, with hexokinase, glucose oxidase (GOx), or glucose-1-dehydrogenase (GDH), as disclosed in Eun-Hyung Yoo & Soo-Youn Lee, “Glucose Biosensors: An Overview of Use in Clinical Practice,” MDPI Sensors 2010 at 4558-4576, referred to elsewhere herein as “Yoo & Lee,” the disclosure of which is incorporated by reference herein in its entirety.

As also shown in FIGS. 1 through 4, glucose sensor 10 may include a cover 18 adapted to conceal and/or protect, for example, substrate 12 and one or more processors 14. Cover 18 may be mounted to substrate 12 by any method, for example, with an adhesive, with mechanical fasteners, with an interference fit, and/or with deflectable tabs or projections. In FIGS. 1 through 4, cover 18 is shown as a substantially transparent material in order to facilitate illustration of aspects of the invention concealed beneath cover 18; however, in some aspects, cover 18 may be made of a translucent or an opaque material.

Though in some aspects cover 18 may comprise a transparent or translucent material, cover 18 may be an opaque material. In some aspects, cover 18 may comprise a metallic, plastic, or elastomeric material. In some aspects, cover 18 may be made from a plastic, for example, a lightweight, moldable plastic. It is envisioned that cover 18 may be made from one or more of the following plastics: a polyamide (PA), for example, nylon; a polyethylene (PE), high-density polyethylene (HDPE); low-density polyethylene (LDPE); a polyethylene terephthalate (PET); a polypropylene (PP); a polyester (PE); a polytetrafluoroethylene (PTFE); a polystyrene (PS); an acrylonitrile butadiene styrene (ABS); a polycarbonate (PC); or a polyvinylchloride (PVC); among other plastics.

In some aspects, cover 18 may be made from an elastomer. An elastomeric cover 18 may be made from a natural polymer, such as polyisoprene rubber, or a synthetic polymer, such as a neoprene; a thermoplastic elastomer (TPE); a thermoplastic rubber; thermoplastic polyurethane; a polyvinyl chloride; a liquid silicone rubber (LSR); or an ethylene propylene diene monomer (EPDM) rubber, and the like.

As shown in FIGS. 3 and 4, for example, cover 18 may include one or more or a plurality of adhesive layers 19, for example, one or more adhesive layers mounted to the lower surface of cover 18, for instance, about the lower perimeter of cover 18. Adhesive layers 19 may be used to mount cover 18 (and its contents) to a surface, for example, at least temporarily mount sensor 10 to the skin (not shown) of a subject. In some aspects, the adhesive of layer 19 may comprise an acrylic adhesive, for example, an acrylic adhesive designed for medical applications and skin contact. In some aspects, the layer 19 may include one or more foam layers, for example, an ethylene vinyl acetate (EVA) foam layer, such as an EVA foam provided by Avery Dennison, for instance, MED 5634 EVA foam, or a similar foam material. In some aspects, the foam layer may have an adhesive on one or both sides of the foam layer, for example, an acrylic adhesive. The acrylic adhesive on or more sides of the foam layer may bond or otherwise adhere to sensor 10 and/or to the target surface.

According to some aspects, as shown in FIGS. 1 through 4, glucose sensor 10 may include one or more power supplies 20, an activation switch 22, one or more data storage devices 24, one or more transmitting devices 26, one or more communication or power ports 28, and/or one or more potentiostats 30, among other conventional electronic components. These components may be mounted to substrate 12, for example, a PCB, by any means. In some aspects, the power supply 20 may include one or more batteries, for example, one or more lithium-polymer (LiPo) batteries, or their equivalent. In some aspects, power supply 20 may include a battery capable of providing 1 to 12 volts DC (VDC), for example, about 3.7 VDC. In some aspects, power supply 20 may provide electric current at from 100 to 1000 milliamp-hours (mAh), for example, about 500 mAh.

As shown in FIGS. 1 through 4, power supply 20 may be electrically connected or coupled to substrate 12 via one or more leads 21 and connector 23. Connector 23 may engage a power port 32 mounted to and electrically coupled to substrate 12. Though any power port may be provided, in some aspects, power port 20 may comprise a connector provided Japanese Solderless Terminal (JST), for example, a PH-type connector, or its equivalent.

Activation switch 22 may comprise any type of activation, or “on-off,” switch adapted to energize and de-energize sensor 10 with power from power supply 20. In some aspects, switch 22 may be a toggle switch, for example, a tactile switch such as a tactile, momentary switch with an activation button 25. In some aspects, the switch 22 may energize the sensor 10 only during tactile activation. In some aspects, switch 22 may energize sensor 10 substantially continually between being toggled on and toggled off. In some aspects, switch 22 may contact cover 18, for example contact the inner surface of cover 18, and may be engaged and disengaged upon deflection of cover 18, for instance, manual deflection or “pressing” by the subject or a healthcare provider. In some aspects, activation button 25 may be accessible through cover 18, for example, through a penetration or hole (not shown) in cover 18. In some aspects, switch 22 may be a tactile, momentary switch provided by Omron Electronics , for example, a B3F-5151 tactile switch, or its equivalent.

Data storage device 24 may comprise any device adapted to at least temporarily store or record the data detected by electrode assembly 15 and/or one or more processors 14. In some aspects, data storage device 24 may be a memory device provided by Secure Digital (SD), for example, a “micro SD”-type card device, or its equivalent. As shown in FIGS. 1 through 4, storage device 24 may comprise a removal “card” received by a cardholder mounted to substrate 12. In some aspects, the capacity of data storage device 24 may range from about 8 gigabytes (Gbytes) to about 256 Gbytes, but in some aspects may have a capacity of at least about 8 Gbytes.

The one or more transmitting devices 26 provided on substrate 12 may comprise any device(s) adapted to transmit signals, for example, signals corresponding to the data detected by electrode assembly 15 and/or one or more processors 14. In some aspects, transmitting device 26 may transmit signals by wire, for example, via one or more leads (not shown). In some aspects, transmitting device 26 may transmit signals wirelessly, for example, employing one or more conventional wireless protocols, such as, the Bluetooth® protocol, Wi-Fi, near-field communication (NFC), and/or radio, among others. In some aspects, the one or more transmitting devices 26 may be a Bluetooth radio module, for example, a module provided by Microchip Technology Inc., for example, an Microchip RN-42 Bluetooth module, or its equivalent.

The one or more communication or power ports 28 provided on substrate 12 may comprise any communication, data, and/or power port. In some aspects, port 28 may be a USB port or a “micro USB” port, as known in the art. One or more ports 28 may be provided to power sensor 10, for example, in place of or in addition to power supply 20, or to recharge power supply 20. One or more ports 28 may be provided to communicate with processor 14, for example, to program or reprogram the software on processor 14. In some aspects, one or more ports 28 may be adapted to transmit or receive electrical signals, for example to download data from sensor 10 and/or to transmit data from sensor 10 to an external receiver and/or to receive data or input from an external transmitter.

In some aspects, the one or more potentiostats 30 may be provided on substrate 20 and may be configured to detect the target substance and/or the target condition in the fluid, though other devices and methods may be used. For instance, in some aspects, potentiostat 30 may provide an output current (I_{O}) reflective or corresponding to the voltage across two electrodes of electrode assembly 15. Potentiostat 30 may be a potentiostat provided by Texas Instruments (TI), for example, a TI LMP91000 series potentiostat, or its equivalent. In some aspects, a potentiostat may not be used, for example, when the detection and/or analysis do not require a potentiostat or its function.

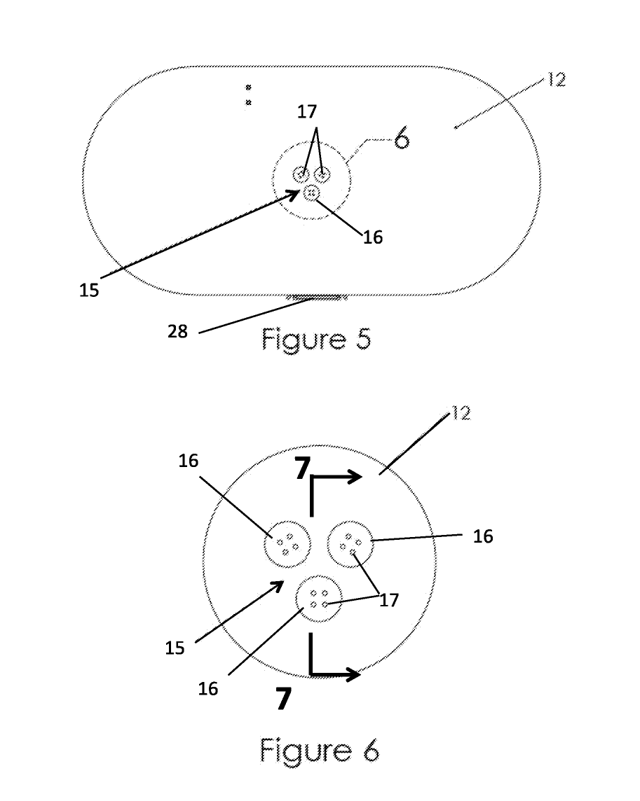

FIG. 5 is a bottom view of substrate 12, for example, a PCB, shown in FIG. 4 according to some aspects of the invention. As shown in FIG. 5, according to some aspects, electrode assembly 15 may include three electrodes 16, and each electrode 16 may include projections or micro-needles 17. In some aspects, micro-needles 17 may extend through, protrude from, mount to, or otherwise extend from substrate 12. In some aspects, for example as shown in FIG. 3, substrate 12 may be a substantial planar substrate including a substantially planar lower surface from which one or more micro-needles 17 extend. According to some aspects, electrode 16 may include any form and/or type of projections or micro-needles 17 adapted to provide the functions disclosed herein. One type of electrode assembly 15 that may be used in some aspects of the invention is shown in FIG. 6.

FIG. 6 is a detailed view of the substrate 12 shown in FIG. 5 as identified by Detail 6 in FIG. 5 showing one set of electrodes 16 including projections or micro-needles 17 that may be used for electrode assembly 15 according to some aspects of the invention. According to some aspects, one or more electrodes 16 may be provided for electrode assembly 15. In some aspects, when a potentiostat 30 is used which requires an input voltage from three electrodes 16, electrode assembly 15 may include three electrodes, for example, a working electrode (WE), a counter electrode (CE), and a reference electrode (RE), as known in the potentiostat art.

FIG. 7 is a detailed cross section of one electrode that may be used for electrode 16 shown in FIGS. 5 and 6 as viewed along section lines 7-7 shown in FIG. 6, according to some aspects of the invention. As shown in FIG. 7, in some aspects, electrode 16 may comprise a cylindrical body 40 mounted in substrate 12 and having one or more projections or micro-needles 17. Cylindrical body 40 may include a surface 41, for example, a lower surface, having micro-needles or other projections 17. In some aspects of the invention, cylindrical body 40 may comprise a metallic material, a plastic material (for example, any one or more of the plastics disclosed herein), or an elastomeric material (for example, any one or more of the elastomeric materials disclosed herein). In some aspects, cylindrical body 40 may comprise an electrically conductive material, for example, a copper, a nickel, steel, a stainless steel, silver, a platinum, a gold, or a conductive polymer as is known in the art, for example, as described by Yoo & Lee.

In some aspects, projections 17 may comprise the same material as cylindrical body 40, for example, projections (or micro needles) 17 may be integrally formed with or machined from the same material. Accordingly, projections 17 and cylindrical body 40 may comprise a single, monolithic construction. In some aspects, projections 17 may be non-integrally formed with cylindrical body 40, for example, projections 17 may be mounted to cylindrical body 17 by any means, such as soldering or an adhesive.

According to some aspects, an electrically conductive material of body 40 may conduct a current or voltage from one or more projections or micro-needles 17 and, for example, conduct the electrical current or voltage to an electrical conductor 42, for example, a wire mounted to or a channel embedded in substrate 12. According to some aspects, conductor 42 may be in electrical communication with other devices on substrate 12, for example, with potentiostat 30 or processor 14. As indicated in FIG. 7, conductor 42 may be mounted to conductive cylindrical body 40 by a bead of solder 43 or other conductive material. In some aspects, electrical conductive body 40 may be in contact with a conductor 44 embedded within substrate 12.

According to some aspects, projections 17 shown in FIG. 7 (and discussed throughout this specification) may be referred to as “micro needles.” For example, projections 17 may be micron-sized, pyramidal-shaped projections that may taper to a relative point and function to contact and, in some aspects, at least partially penetrate a target surface against which projections 17 are contacted, for example, the skin of a subject. However, projections 17 may comprise any projection, pointed or non-pointed, from surface 41 of body 40 that isolates the area of contact of body 40 (and other bodies disclosed herein) with the target surface. In some aspects, projections 17 may comprise one or more projections or micro needles having one or more electrical contacts or “electrodes,” for example, two or more electrical contacts, mounted (for example, deposited) on a single projection or micro needle. The two or more contacts may be in electrical communication with one or more components on substrate 12, for example, with potentiostat 30 or processor 14.

In some aspects of the invention, projections 17 may comprise “micro needle”-type structures, for example, pyramidal structures such as 3-, 4-, 5- or more-sided pyramidal structures, extending from body 40 and tapering to a relative pointed apex of the pyramidal structure. The width of the base and the height of the pyramidal structures of micro-needles 17 may range from about 100 micrometers (um) to about 2000 um, but in some aspects may range from about 400 um and about 1000 um. The lateral spacing between or “pitch” of projections 17 may range from about 500 um to about 2000 um, but in some aspects may range between about 1000 um and about 1500 um, for example, about 1200 um.

In some aspects, projections 17 may not be pyramidal, but may be parallelopipedal, cuboid, hemispheroidal, or ovoid, among other shapes, and may provide the isolated contact with the mating target surface. In some aspects, projections 17 may not taper to an actual “point,” as suggested by the figures, but, due to manufacturing tolerances, may terminate at a distal extremity of small dimension, for example, projections 17 may taper, but may present a generally planar or hemispherical surface against the target surface. Those of skill in the art may envision other shapes of projections 17 without detracting from the function of projections 17.

In some aspects, projections 17 may be elongated, cylindrical projections, for example, much like a conventional “hypodermic needle.” In some aspects, the projections 17 may be elongated, cylindrical projections having a circular, an elliptical, or a polygonal cross section, such as, triangular, square, or rectangular cross section. In some aspects, the elongated cylindrical projections 17 may be hollow, for example, defining an internal lumen, or non-hollow or solid projections. For example, projections 17 may be elongated, cylindrical projections having an aspect ratio, that is, ratio of length to width or diameter, of at least 5, but may have an aspect ratio of about 10 to about 200, for example, about 50 to about 100. In some aspects, the aspect ratio of projections 17 may vary, for example, projections 17 may be elongated, cylindrical projections that taper, for example, from a larger first width or diameter to a smaller second width or diameter. For example, the smaller width or diameter may approach zero, where the projection comes substantially to “a point,” as known in the art. In some aspects of the invention, projections 17 may comprise carbon “nanotubes.” In some aspects, as referenced above, the elongated, cylindrical projections 17 may include one or more electrical contacts or “electrodes,” as disclosed herein, for example, two or more electrical contacts, spaced along the projection, for example, equally spaced.

In some aspects, projections 17 may be omitted, and at least partial contact between surfaces 41 of body 40 with a target surface may be sufficient to provide the contact with the target surface. For example, surface 41 may extend beyond the surface of substrate 12 and provide the contact with the target surface.

According to some aspects, for example as shown in FIG. 7, conductive cylindrical body 40 may facilitate fabrication and/or assembly of electrode 16 in substrate 12. For example, some aspects may provide a conductive cylindrical body 12 including micro-needles 17 that may facilitate establishing electrical communication between micro-needles 17 and other devices on substrate 12, for example, via conductor 42. In some aspects, conductive body 40 including micro-needles or projections 17 previously formed or provided and body 40 may be positioned in substrate 12, for example, positioned in a penetration or through hole in substrate 12. After positioning body 40 with micro-needles 17 in substrate 12, conductor 42 may be connected to body 40 by, for example, soldering solder bead 43 to conductor 42 and body 40.

According to some aspects, cylindrical body 40 may be mounted to substrate 12 by any means, for example, with an adhesive. In some aspects, cylindrical body 40 may include one or more features that may enhance the engagement of cylindrical body 40 to substrate 12, for example, to minimize or prevent rotation of body 40 in substrate 12 or the deflection, separation, or disengagement of body 40 from substrate 12. In some aspects, one or more annular rings 45, as shown in FIG. 7, adapted to engage an annular recess in substrate 12 may be provided. In some aspects, cylindrical body 40 may include one or more annular recesses adapted to engage an annular projection in substrate 12. In some aspects, body 40 and/or substrate 12 may include cooperating structures, such as, keys, or keyways, to minimize or prevent relative rotation of body 40 within substrate 12. (See FIGS. 18 and 19, for examples.)

In some aspects, cylindrical body 40 of substrate 12 may be mounted in a resilient or elastomeric collar or annulus 61, as indicated in phantom in FIG. 7. FIG. 7A is a top plan view of the electrode 16 shown in FIG. 7 including an elastomeric collar or annulus 61 according to some aspects. In FIG. 7A, only a representative portion of substrate 12 is shown. In some aspects, the cylindrical body 40 (or any cylindrical body disclosed herein) of electrode 16 may be mounted in flexible, elastomeric collar 61, which may be mounted in substrate 12. The flexibility or resiliency of collar 61 may provide at least some flexibility to the mounting of electrode 16 in substrate 12 wherein electrode 16 may deflect under load, for example, in contrast to the rigidity of substrate 12, and thus better conform to the surface contacted by electrode 16, for example, the irregular surface of the skin of a heel of a foot of a neonate. In some aspects, collar 61 may be made of any one or more of the elastomeric materials disclosed herein, for example, elastomeric collar 61 might be made from a thermoplastic elastomer (TPE), a thermoplastic urethane, or a silicone rubber. As shown in FIG. 7A, conductor 42 may me attached to electrode 16 in a fashion similar to that shown and described with respect to FIG. 7, for example, with a bead of solder 43.

Though illustrated in FIG. 7A as a hollow, substantially circular cylindrical collar, collar 61 may assume any cylindrical shape, including elliptical cylindrical, rectangular cylindrical, and/or polyhedral cylindrical, among other cylindrical shapes (for example, any one of the cylindrical shapes illustrated tin FIGS. 8 through 17). In addition, though the outside width or outside diameter of collar 61 is shown about twice the outside diameter of cylindrical body 40 in FIG. 7A, the outside width or outside diameter of collar 61 may range from 1.5 times to 10 times the outside diameter or width of cylindrical body 40. For example, in some aspects, the outside diameter or outside width of elastomeric collar 61 may be about 2 to 10 times the outside width or outside diameter of cylindrical body 40, for example, about 3 times the outside width or outside diameter of cylindrical body 40. In some aspects, cylindrical body 40 of substrate 12 may be mounted in a resilient portion of substrate 12. For example, instead of mounted in an elastomeric collar or annulus 61, one or more cylindrical bodies 40 may be mounted in an at least an elastomeric portion of substrate 12, for example, a portion of substrate 12 that may comprise at least 25% of the surface area of substrate 12; or at least 50% of the surface area; or at least 75% of the surface area of substrate 12. In some aspects, the entire area of substrate 12 may substantially comprise an elastomeric material, for example, substrate 12 may comprise a sheet of elastomeric material within which one or more cylindrical bodies 40 having one or more contacts or microneedles 17 may be positioned.

According to some aspects, body 40 of electrode 16 may take any form of cylindrical or non-cylindrical shape. For example, in some aspects, body 40 may not be strictly “cylindrical,” while providing the functions disclosed herein. For instance, body 40 may simply comprise a “body,” that is, a 3-dimensional structure or form of any unspecified shape, or a 3-dimensional structure or form having a well-defined shape, such as, an “hour-glass” shape, and the like. Body 40, and any of the corresponding bodies disclosed herein, may also contain recesses or voids, for example, may be substantially hollow, while providing the functions disclosed herein. FIGS. 8 through 17 illustrate some of the shapes of body 40 envisioned for some aspects.

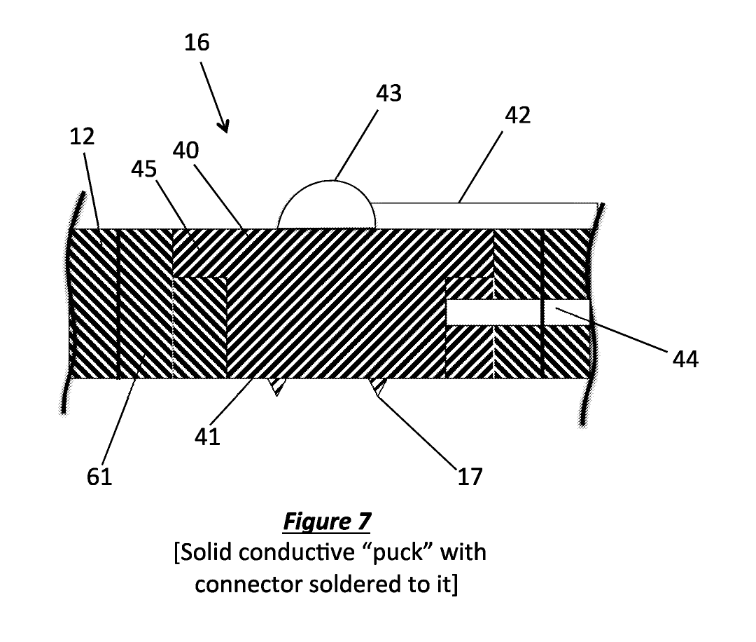

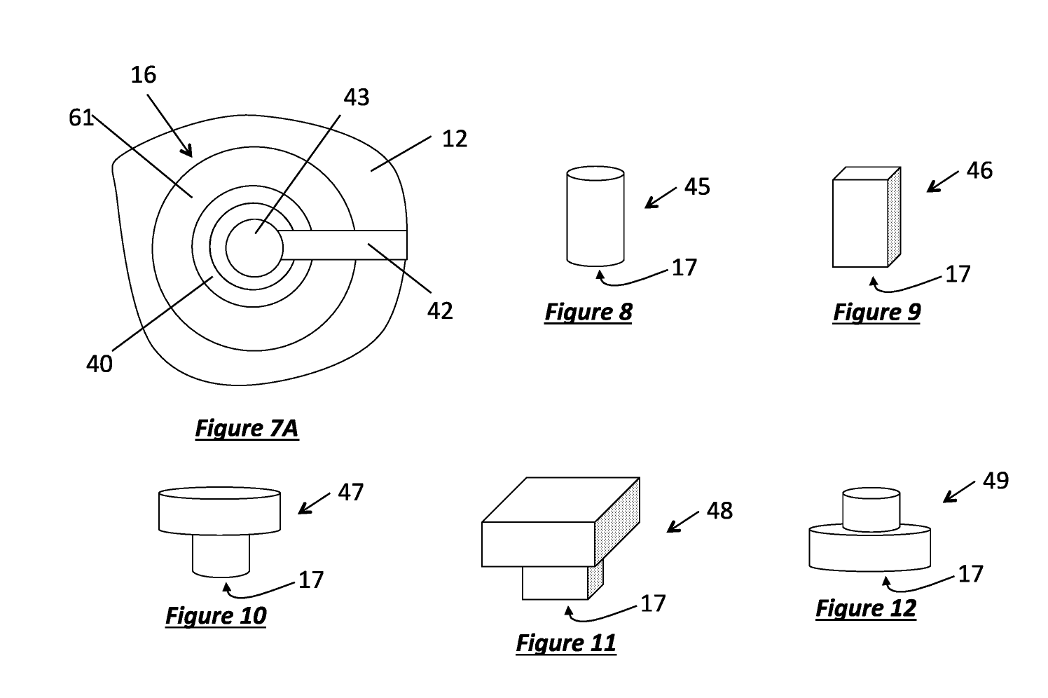

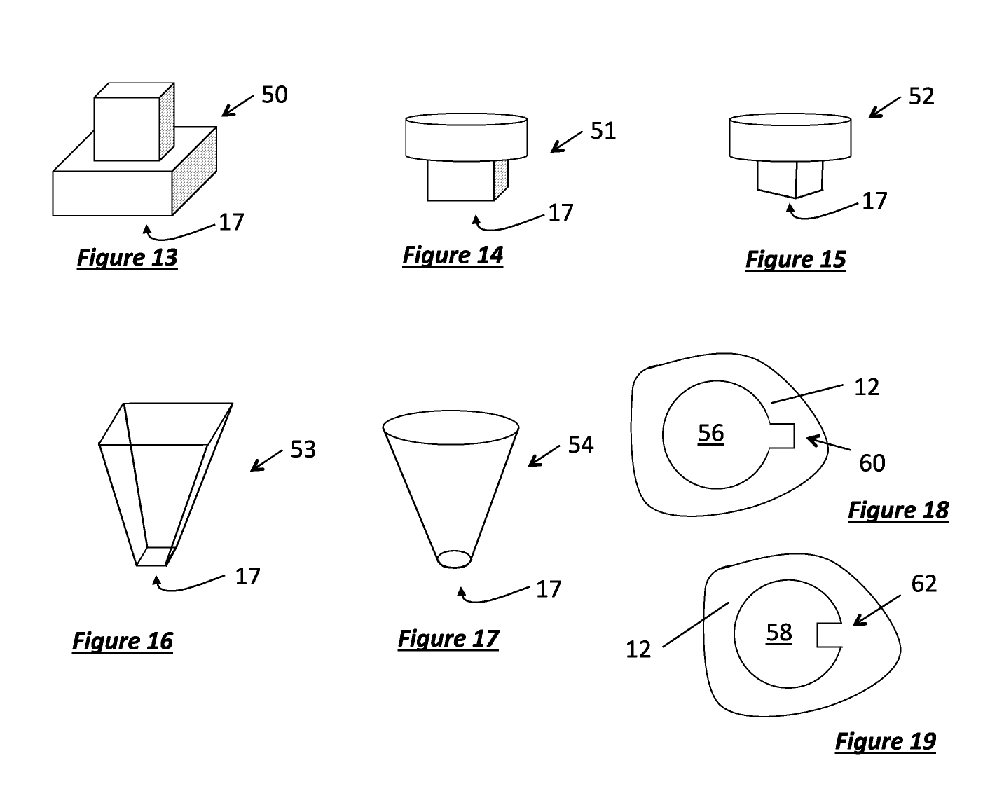

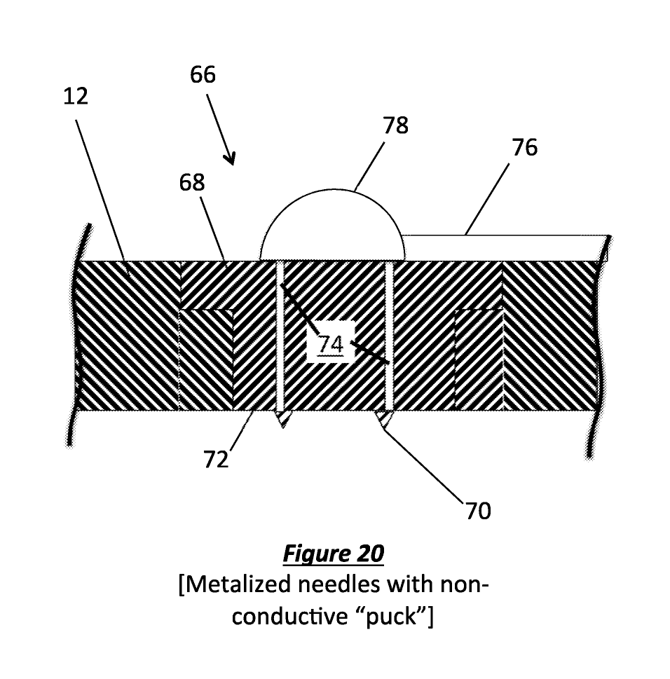

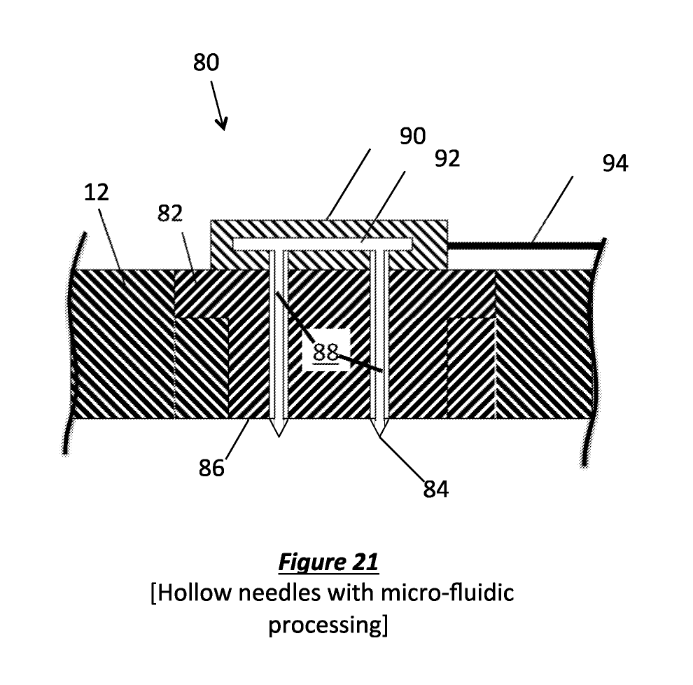

FIG. 8 is a perspective view of circular cylindrical body 45 including one or more projections or micro-needles 17 that may be used for body 40 according to some aspects. FIG. 9 is a perspective view of polygonal cylindrical body 46 including one or more projections or micro-needles 17 that may be used for body 40 according to some aspects. FIG. 10 is a perspective view of a cylindrical body 47 including two or more portions with two or more diameters, and one or more projections or micro-needles 17 (on the smallest diameter portion) that may be used for body 40 according to some aspects. FIG. 11 is a perspective view of a polygonal cylindrical body 48 including two or more portions with two or more widths, and one or more projections or micro-needles 17 (on the smallest width portion) that may be used for body 40 according to some aspects. FIG. 12 is a perspective view of a circular cylindrical body 49 including two or more portions with two or more diameters, and one or more projections or micro-needles 17 (on the largest diameter portion) that may be used for body 40 according to some aspects. FIG. 13 is a perspective view of a polygonal cylindrical body 50 including two or more portions with two or more widths, and one or more projections or micro-needles 17 (on the largest width portion) that may be used for body 40 according to some aspects. FIG. 14 is a perspective view of a cylindrical body 51 including circular portion and a polygonal portion, and one or more projections or micro-needles 17 (on the polygonal portion) that may be used for body 40 according to some aspects. FIG. 15 is a perspective view of a cylindrical body 52 including a circular portion and a triangular portion, and one or more projections or micro-needles 17 (on the triangular portion) that may be used for body 40 according to some aspects. FIG. 16 is a perspective view of a conical cylindrical body 53 including a square, rectangular, or polygonal cross section, and one or more projections or micro-needles 17 (on the smaller or the larger end surface) that may be used for body 40 according to some aspects. FIG. 17 is a perspective view of a conical cylindrical body 52 including a circular or elliptical cross section, and one or more projections or micro-needles 17 (on the smaller or the larger end surface) that may be used for body 40 according to some aspects.

FIGS. 18 and 19 are top views of a cylindrical body 56 and a cylindrical body 58, respectively, as positioned in substrate 12 having one or more projections or micro-needles 17 (not shown) that may be used for body 40 according to some aspects. Cylindrical bodies 56 and 58 may have a key-type structure 60 and keyway-type structure 62, respectively, which may cooperate with complementary structures in substrate 12 that may be used to minimize or prevent rotation of any one the cylindrical bodies disclosed herein, for example, as shown and described with respect to FIGS. 7 through 17.

FIG. 20 is a detailed cross section, similar to FIG. 7, of an electrode assembly 66 that may be used for electrode assembly 16 shown in FIGS. 5 and 6, according to some aspects. As shown in FIG. 20, in some aspects, electrode 66 may comprise a cylindrical body 68 mounted in substrate 12 and including projections or micro-needles 70. Cylindrical body 68 may include a surface 72, for example, a lower surface, including micro-needles or other projections 70. In some aspects, cylindrical body 68 may comprise a metallic material, a plastic material (for example, any one or more the plastics disclosed herein), or an elastomeric material (for example, any one or more of the elastomeric material disclosed herein). In some aspects, cylindrical body 68 may comprise a non-conductive material, for example, a plastic or an elastomer. Projections or micro-needles 70 may include some form of electrical conductive material, for example, projections 70 may comprise a conductive metallic material embedded in or otherwise mounted to, for example, non-conducting body 68. In some aspects, projections 70 may be coated, impregnated, or otherwise treated with a conductive material, such as a metal coating, for instance, projections 70 may be “metalized.” In some aspects, projections 70 may be treated by sputter coating, electron beam physical vapor deposition, vacuum vapor deposition, electrolytic deposition, and/or metallic nanoparticle deposition to metalize projections 70.

As shown in FIG. 20, in some aspects, the “metalized” projections 70 may be electrically coupled and/or electrically communicate with one or more devices on substrate 12 via one or more conductors 74 and one or more conductors 76. As indicated in FIG. 20, conductor 76 may connect with one or more conductors 74 via a bead of solder 78 or other conductive material. In some aspects, the one or more conductors 74 may be positioned in and pass through body 68, for example, a non-conductive material. In some aspects, conductors 74 connecting metalized projections or micro-needles 70 to other devices on substrate 12 may pass about the periphery of body 68 or through or along substrate 12.

FIG. 21 is a detailed cross section, similar to FIGS. 7 and 20, of an electrode 80 that may be used for electrode assembly 16 shown in FIGS. 5 and 6, according to some aspects. As shown in FIG. 21, electrode 80 may comprise a cylindrical body 82 mounted in substrate 12 and including projections or micro-needles 84. Cylindrical body 82 may include a surface 86, for example, a lower surface, having micro-needles or other projections 84. In some aspects, cylindrical body 82 may comprise a metallic material, a plastic material (for example, any one or more the plastics disclosed herein), or an elastomeric material (for example, any one or more of the elastomeric materials disclosed herein). Projections or micro-needles 84 may comprise some form of electrical conductive material, for example, be metalized, as discussed herein, and may electrically communicate with one or more devices on substrate 12 as disclosed herein. In some aspects, as shown FIG. 21, projections or micro-needles 84 may be adapted to capture at least some fluid, for example, a gas or a liquid, from the target surface or from beneath the target surface.

In some aspects, the one or more projections 84 may be in fluid communication with the one or more passages 88, for example, conduits, through-holes, or lumens, which may extend through cylindrical body 82. In some aspects, passages 88 may be through holes at least partially extending through cylindrical body 82. In some aspects, passages 88 may be conduits extending at least partially through cylindrical body 82. In some aspects, passages 88 may comprise conduits that are integral with projections or micro-needles 84, for example, projections or micro-needles 84 may comprise the distal ends of conduits 88. In some aspects, passages 88 may comprise a hollow conduit having a distal end 84 that extends beyond the surface 86 of cylindrical body 82, for example, passages 88 may comprise an integral “needle” having an internal lumen 88 and a pointed end 84. Passages 88 may be circular, elliptical, or polygonal, in cross section. For example, passages 88 may be square or rectangular in cross section.

In some aspects, the inside width or insider diameter of passages or lumens 88 may range from about 0.010 millimeters [mm] (that is, 10 microns) to about 2.0 mm, for example, about 0.050 mm (that is, 50 microns).

As shown in FIG. 21, passages 88 may be in fluid communication with a detection and/or analysis device 90 mounted to substrate 12 and/or to cylindrical body 82. In some aspects, detection or analysis device 90 may be at least partially embedded within cylindrical body 82, for example, positioned in, or encased in cylindrical body 82. Detection or analysis device 90 may include an internal cavity 92 adapted to receive a fluid transferred through passages 88 from projections 84. In some aspects, internal cavity may be an internal cavity of cylindrical body 82.

In some aspects, analysis device 90 may be adapted to interact with the fluid received via passages 88 and output an electrical signal via connector 94 to another device, for example, to another device mounted to substrate 12, for instance, to a processor 14. Device 90 may be a micro-fluidic device, for example, a micro fluidic device comprising a micro electro-mechanical system, that is, a “MEMS” device.

In some aspects, device 90 may be adapted to detect at least one substance and/or at least one condition of the fluid introduced by passages 88, for instance, an analyte present in a gas or liquid. The signal transmitted by conductor 94 may correspond to or be associated with the substance and/or condition detected by device 90.

The substances that may be detected by device 90 may be glucoses, but are not limited to glucoses. For example, in some aspects of the invention device 90 may be adapted to detect alcohols, ions, metabolites, proteins, salts, fats, sugars, lipids, enzymes, amino acids, nucleotides, genes, antioxidants, organic acids, drugs, narcotics, pharmaceuticals, chemicals (such as, tetrahydrocannabinol (THC) or THC metabolites, or anti-bodies to THC), vitamins, melatonin, electrolytes, carbon dioxide, carbon monoxide, among others. The detection of one or more of these substances may be practiced alone, or in conjunction with the voltage differential detected by projections 84.

Also, device 90 may be adapted to detect one or more of the following conditions of the fluid: temperature, humidity, conductivity, resistivity, pH, alkalinity, acidity, and viscosity, among others. The detection of any one or more of these conditions may be practiced alone, or may be practiced while detecting one or more substances. The detection of one or more of these conditions may be practiced alone, or in conjunction with the voltage differential detected by projections 84. In some aspects, device 90 may be adapted to detect and/or analyze any desired substance and/or condition in the fluid collected by projections 84, for example, an immunoassay, aptamer ranges, polymer chain reaction (PCR) analysis, optical sensing, and/or an electrochemical sensing, such as, a fluorometric sensing and/or the amperometric sensing disclosed in Grace Wu & Muhammad Zaman, “Amperometric measurements of ethanol on paper with a glucometer,” Talanta 134 (2015) at 194-199, the disclosure of which is included by reference herein in its entirety.

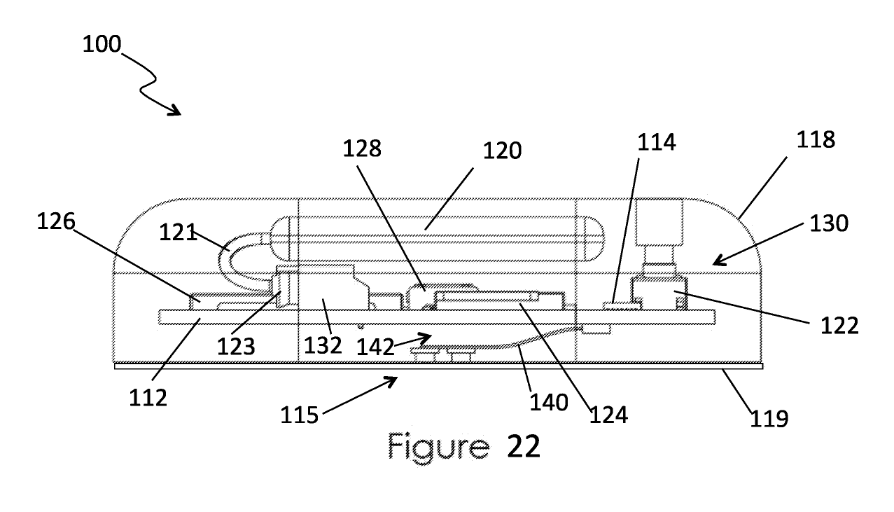

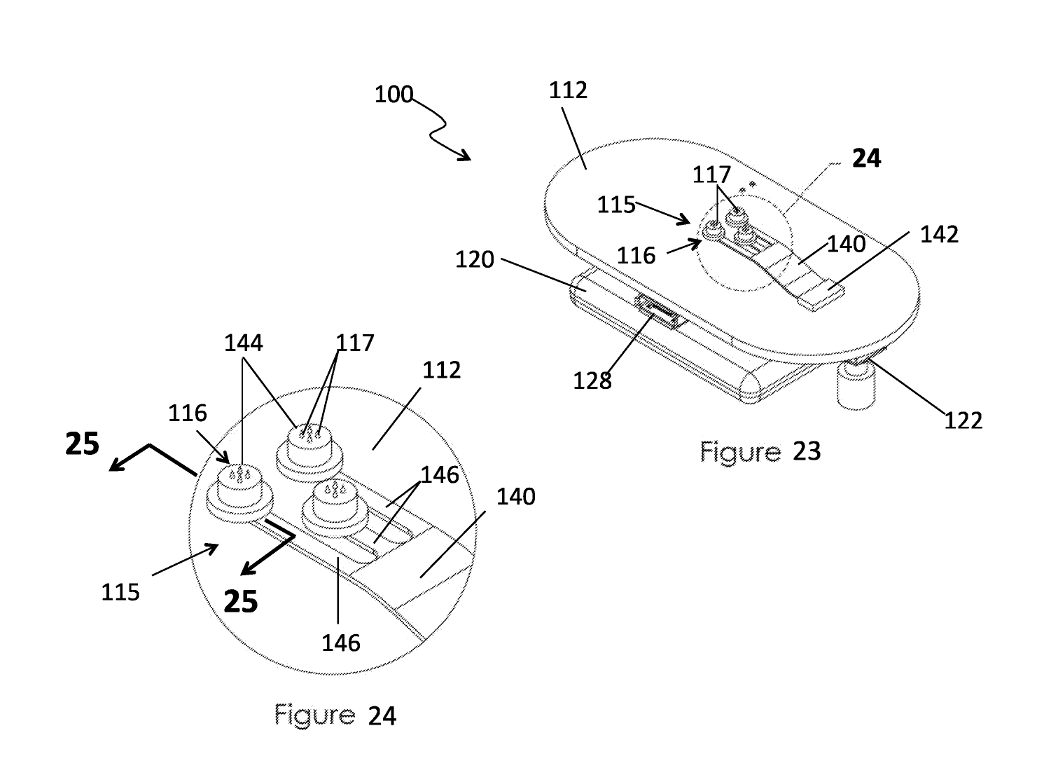

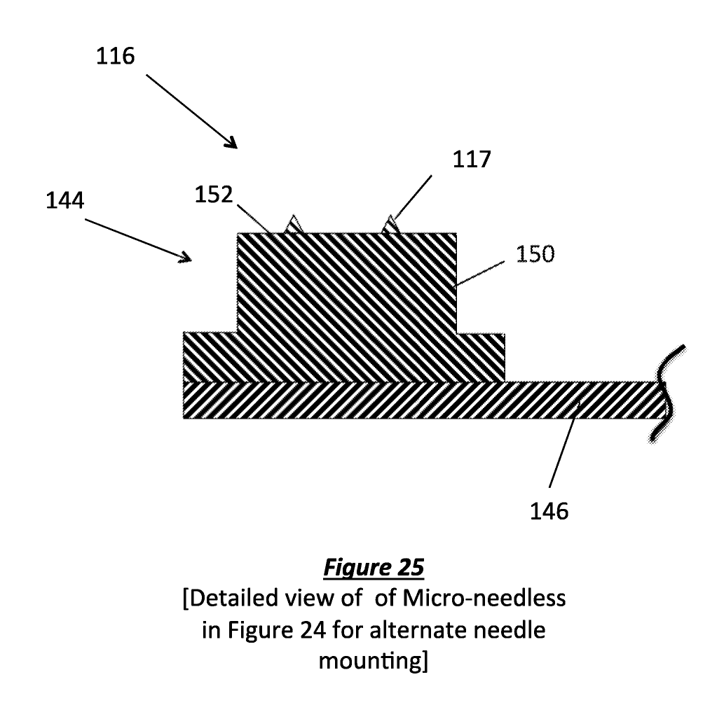

FIG. 22 is a side elevation view of another glucose sensor 100 according to some aspects. FIG. 23 is a bottom perspective view of the glucose sensor 100 shown in FIG. 22, with cover removed to facilitate illustration of some features. In a fashion similar to sensor 10 shown in FIGS. 1 through 7, glucose sensor 100 may include a substrate 112, one or more processors 114 mounted to substrate 112, and an electrode assembly 115 with electrodes 116 having a plurality of micro-needles 117 mounted to the substrate 112 and operatively connected to the one or more processors 114. In some aspects, substrate 112 may have all the features and attributes of substrate 12 disclosed herein, for example, substrate 112 may comprise a PCB. As disclosed herein, substrate 112 may include at least some elastomeric material. For example, a portion of substrate 112 may comprise an elastomeric material, such as elastomeric collar 61 disclosed herein. FIG. 24 is a detailed view of electrode 116 shown in FIG. 23 as identified by Detail 24 in FIG. 23.

The one or more processors 114 mounted to substrate 112 may have all the features and attributes of processor 114 disclosed herein. For example, processors 114 may comprise any processor adapted to contain software, for example, executable software, and receive and transmit electrical signals from electrode 116.

The electrode 116 of glucose sensor 100 may have all the features and attributes of electrode 16 disclosed herein. For example, electrode 116 may include any one or more micro-needles 117 adapted to perform the desired functions.

Glucose sensor 100 shown in FIGS. 22, 23, and 24 may also include a cover 118 adapted to conceal and/or protect, for example, substrate 112 and one or more processors 114. Cover 118 may have the features and attributes of cover 18 disclosed herein. Glucose sensor 100 may include one or more adhesive layers 119, for example, an adhesive layer having all the features and attributes of adhesive layer 19 disclosed herein; one or more power supplies 120 having all the features and attributes of power supply 20 disclosed herein; an activation switch 122 having all the features and attributes of activation switch 22 disclosed herein; one or more data storage devices 124 having all the features and attributes of data storage device 24 disclosed herein; one or more transmitting devices 126 having all the features and attributes of transmitting device 26 disclosed herein; one or more communication or power ports 128 having all the features and attributes of ports 28 disclosed herein; and/or one or more potentiostats 130 (not shown, but its location is generally indicated by arrow 130 in FIG. 22) having all the features and attributes of potentiostat 30 disclosed herein, among other conventional electronic components. These components may be mounted to substrate 112, for example, a PCB, by any means.

As shown in FIG. 22, power supply 120 may be electrically connected to substrate 112 via one or more leads 121 and connector 123 which may engage a power port 132 mounted to and electrically coupled to substrate 112. Leads 121, connecter 123, and port 132 may be similar to the leads 21, connecter 23, and port 32, respectively, disclosed herein.

In some aspects, such as shown in FIGS. 22, 23, and 24, in contrast to sensor 10 shown in FIGS. 1 through 7, sensor 100 may include an electrode assembly 115 mounted distally, or displaced from, substrate 112, for example, mounted below substrate 112 as shown in FIG. 22. In the aspects shown in FIGS. 23 and 24, electrode assembly 115 may be mounted to substrate 112 by one or more flexible or deflectable projections, tabs, or “fingers” 140. The one or more flexible projections 140 may provide a resilient or deflectable mounting to electrode 116. In some aspects, the resilient or deflectable mounting of electrode 116 to substrate 112 may promote or encourage contact of electrode 116 and the target surface (not shown) to which sensor 110 is mounted, for example, the surface of the skin of a subject. In some aspects, resilient projection 140 may be made from a metallic material, a plastic material, or an elastomeric material, and may provide the contact with the target surface.

In some aspects, for example as shown in FIGS. 23 and 24, projection 140 may be substantially rectangular in cross section. However, in some aspects, projection 140 may have any cross section, for example, a circular cross section, an elliptical cross section, or a polygonal cross section, such as a square cross section, among others.

According to some aspects, for example as shown in FIGS. 23 and 24, electrode 116 may communicate electrically with a component on substrate 112, for instance, to processor 114, by any means, for example, with one or more leads or wires from electrode 116 to the component. The one or more leads or wires (not shown) may pass from electrode 116 to a component on substrate 112 via any path, for example, extend freely across the void 142 (see FIG. 22) between electrode assembly 115 and substrate 112 and/or along, on, or through the one or more projections 140. In some aspects, projection 140 may comprise an electrically conductive material, for example, copper or aluminum, and may provide electrical communication between electrode 116 and one or more components on substrate 112.

In some aspects, projection 140 may comprise a “ribbon cable,” for example, a flexible or resilient ribbon cable, and may provide a conduit for transmitting of electrical signals from electrode 116 to substrate 112. As indicated in FIGS. 23 and 24, in some aspects, projection 140 may communicate with substrate 112 via connector 142, for example, a ribbon cable connector or its equivalent. In some aspects, projection 140 may be mounted to substrate 112 by connector 142.

As shown in FIG. 24, electrode 116 may include one or more projection or needle holders 144 including one or more individual projections or needles 117 or micro-needles. For example, in some aspects, the projections 117 may be integrally or monolithically formed with a needle holder 144. Needle holders 144 may be mounted to projection 140, for example, by any means, for instance with an adhesive or by solder or welding. In some aspects, for example as shown in FIG. 24, three needle holders 144 may be provided in electrode 116, and each needle holder 144 may include four projections or needles 117. In some aspects, one or more needle holders 144 may be provided, for example, three or more needle holders 144, or 10 or more needle holders 144. In some aspects, needle holders 144 may retain one or more projections or needles 117, for example, two or more needles 117, five or more needles 117, 10 or more needles, or 50 or more needles 117. In some aspects, needle holders 144 may not be used, and needles 117 may be mounted to projection 140, for example, mounted to one or more projections 146. In some aspects, needles 117 may be mounted directly to projection 140 or projections 146, for example, formed on or integrally formed with projection 140 or projections 146, for instance, formed of the same conductive material.

As shown in FIG. 24, needle holders 144 may be mounted to smaller projections 146, for example, individual “finger” projections, or “fingers,” that may be mounted to or project from projection 140. In some aspects, the fingers 146 may vary in length or may be of substantially the same length, for example, depending upon the desired spacing and/or alignment of needle holders 144 and/or micro-needles 117. In some aspects, needle holders 144 may be positioned in “staggered” position relative to other needle holders 144, for example, where staggered may comprise having a centerline spaced from the centerline of one or more other needle holders 144. In some aspects, needle holders 144 may be “aligned” with one or more other needle holders 144, for example, where aligned may comprise having a centerline substantially collinear with a centerline of one or more other needle holders 144.

According to some aspects, one or more fingers 146 may be provided with the one or more projections 140. Projections 140 and fingers 146 may be circular, oval, square, or polygonal in cross section and may be made from any one or more of the materials disclosed herein, for example, metallic, plastic, or elastomeric. In some aspects, projections 140 and fingers 146 may be metallic, specifically, an electrically conductive metal that can electrically couple projections 117, holders 144, projections 146, and projection 140 to devices on substrate 112, for example, to potentiostat 130

FIG. 25 is a detailed cross section view, similar to FIGS. 7, 20, and 21, of electrode 116 shown in FIGS. 23 and 24 as viewed along section lines 25-25 shown in FIG. 24, according to some aspects. As shown in FIG. 25, in some aspects, electrode 116 may comprise a needle holder 144 having cylindrical body 150 mounted to projection 146 and including projections or micro-needles 117. Cylindrical body 150 may include a surface 152, for example, a lower surface, having micro-needles or projections 117. In some aspects, cylindrical body 150 may comprise a metallic material, a plastic material (for example, any one or more the plastics disclosed herein), or an elastomeric material (for example, any one or more of the elastomeric materials disclosed herein). In some aspects, cylindrical body 150 may comprise an electrically conductive material, for example, copper, silver, or a conductive polymer. According to some aspects, an electrically conductive material of body 150 may conduct a current or voltage from projections or micro-needles 117 or conduct the electrical current or voltage to an electrical conductor (not shown), for example, a wire mounted to or a channel embedded in projection 146, for instance, to place projection 146 in electrical communication with other devices on substrate 112.

In some aspects, cylindrical body 150 may be non-electrically conductive, and projections or micro needles 117 may be conductive or metallized and may be in electrical communication with projection 146 and devices on substrate 112, as disclosed herein.

Cylindrical body 150 of needle holder 144 may assume any one of the shapes of the cylindrical bodies disclosed herein, for example, those shown in FIGS. 8 through 19, among others.

According to some aspects, the size of sensors 10 and 100 (and any sensors disclosed herein) may vary depending upon the nature and environment of their intended use. For example, in some aspects, sensors 10 and 100 may have lengths ranging from about 10 mm to about 500 mm. In some aspects, sensors 10 and 100 may have lengths ranging from about 50 mm to about 100 mm, for example, about 70 mm. In some aspects, sensors 10 and 100 may have widths ranging from about 5 mm to about 200 mm. In some aspects, sensors 10 and 100 may have widths ranging from about 20 mm to about 60 mm, for example, about 40 mm. In some aspects, sensors 10 and 100 may have thicknesses or heights ranging from about 5 mm to about 100 mm. In some aspects, sensors 10 and 100 may have heights ranging from about 10 mm to about 20 mm, for example, about 15 mm.

The shapes of sensors 10 and 100 (and any sensors disclosed herein) may vary depending upon the nature and environment of their intended use. For example, though sensors 10 and 100 are shown herein as generally rectangular in shape and having rounded corners, sensors 10 and 100 may be circular, oval, square (for example, with rounded corners), or polygonal (for example, with rounded corners) in shape while providing the functions and benefits disclosed herein.

While various embodiments have been described above, it should be understood that they have been presented by way of example and not limitation. It will be apparent to persons skilled in the relevant art that various changes in form and detail can be made therein without departing from the spirit and scope. After reading the above description, it will be apparent to one skilled in the relevant art how to implement alternative embodiments.

In addition, it should be understood that any figures, which highlight the functionality and advantages, are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A glucose sensor comprising:
a substrate;
a processor mounted to the substrate;
a plurality of electrodes mounted to the substrate and operatively connected to the processor, the plurality of electrodes adapted to detect a condition on or below a surface.

2. The glucose sensor as recited in claim 1, wherein each of the plurality of electrodes comprises at least one projection.

3. The glucose sensor as recited in claim 2, wherein the at least one projection comprises a plurality of micro-needles.

4. The glucose sensor as recited in claim 1, wherein each of the plurality of electrodes comprises a cylindrical body.

5. The glucose sensor as recited in claim 4, wherein the cylindrical body comprises at least one projection.

6. The glucose sensor as recited in claim 4, wherein the cylindrical body comprises at least one of a circular cylindrical body, an elliptical cylindrical body, and a polygonal cylindrical body.

7. The glucose sensor as recited in claim 4, wherein each of the plurality of electrodes comprises an elastomeric collar retaining the cylindrical body.

8. The glucose sensor recited in claim 7, wherein the elastomeric collar comprises at least one of a thermoplastic elastomer [TPE], a thermoplastic urethane [TPU], and a liquid silicone rubber [LSR].

9. The glucose sensor as recited in claim 1, wherein the sensor further comprises a potentiostat mounted to the substrate, wherein the potentiostat is electrically coupled to the plurality of electrodes.

10. The glucose sensor as recited in claim 1, wherein the plurality of electrodes is distally mounted to the substrate.

11. The glucose sensor as recited in claim 10, wherein the plurality of electrodes is distally mounted to the substrate by a flexible projection.

12. The glucose sensor as recited in claim 11, wherein the flexible projection comprises a flexible, electrically conductive projection.

13. The glucose sensor recited in claim 11, wherein the flexible projection is metallic.

14. The glucose sensor recited in claim 13, wherein the metallic flexible projection comprises one of metallic micro-needles and metalized micro-needles.

15. The glucose sensor recited in claim 1, wherein the sensor further comprises a transmitter mounted on the substrate and operatively connected to the processor, the transmitter adapted to transmit at least one signal to at least one external receiver.

16. The glucose sensor recited in claim 1, wherein the glucose sensor comprises a neonatal glucose sensor.

17. The glucose sensor recited in claim 1, wherein the glucose sensor comprises one of a glucose sensor adapted to continuously and intermittently sense glucose.

18. The glucose sensor recited in claim 1, further comprising an adhesive layer adapted to at least temporarily adhere to a target surface.

19. The glucose sensor recited in claim 18 wherein the adhesive layer comprises a plurality of adhesive layers.

20. The glucose sensor recited in claim 5, wherein the at least one projection is integrally formed with the cylindrical body.

21. A method for detecting glucose in a subject, the method comprising:
contacting the skin of the subject with at least two electrically conductive projections;
sensing an electrical signal between the at least two electrically conductive projections;
relating the sensed electrical signal to a glucose concentration; and
transmitting the glucose concentration to a receiver.

22. The method as recited in claim 21, wherein the method further comprises allowing the at least one of the at least two electrically conductive projections to deflect when contacting the skin of the subject.

23. The method as recited in claim 22, wherein allowing at least one of the at least two electrically conductive projections to deflect comprises mounting the at least one projection of the at least two electrically conductive projections in an elastomeric collar.

24. The method as recited in claim 22, wherein allowing the at least one of the at least two electrically conductive projections to deflect comprises mounting the at least one projection of the at least two electrically conductive projections on a flexible projection.

25. The method as recited in claim 21, wherein the method further comprises collecting a fluid sample from the subject.

26. The method as recited in claim 25, wherein the at least two electrically conductive projections comprise hollow projections, and wherein collecting the fluid sample from the subject comprises collecting fluid through the hollow projections.

27. The method as recited in claim 21, wherein the at least two electrically conductive projections comprise a plurality of electrically conductive micro-needles.

28. The method as recited in claim 21, wherein the subject comprises a neonate.
